(19) United States
(12) Patent Application Publication
(10) Pub. No.: US 2022/0089662 A1
(43) Pub. Date: Mar. 24, 2022

(54) PREPRATION METHOD AND APPLICATION OF ANTI-INFLAMMATORY KIDNEY PROTECTING CLAM PEPTIDE

(71) Applicants: Chenland Nutritionals Inc., Irvine, CA (US); Qingdao Chenland Biological Technolgy Co., Ltd., Qingdao (CN)

(72) Inventors: Shengcan Zou, Qingdao (CN); Huihui Ren, Qingdao (CN); Jinli Liu, Qingdao (CN); Tingting Liu, Qingdao (CN); Shanglong Wang, Qingdao (CN); Tingfeng Cao, Qingdao (CN); Jiancheng Zong, Qingdao (CN)

(21) Appl. No.: 17/533,444

(22) Filed: Nov. 23, 2021

Related U.S. Application Data
(63) Continuation of application No. PCT/CN2021/116427, filed on Sep. 3, 2021.

(30) Foreign Application Priority Data
Sep. 4, 2020 (CN) 202010922422X
Aug. 27, 2021 (CN) 2021109973209

(51) Int. Cl. C07K 14/435; C07K 1/36; C12P 21/06

(52) U.S. Cl. CPC C07K 14/43504; C12P 21/06; C07K 1/36

(57) ABSTRACT

A preparation method and application of an anti-inflammatory and kidney-protecting clam peptide are disclosed. The method includes the steps of: cleaning and crushing the whole meat of a HonDau clam to obtain serous fluid, adding compound protease accounting for 0.1-0.3% of the weight of the serous fluid, carrying out enzymolysis, centrifugation, membrane separation and purification, and spray drying to obtain clam peptide powder. The clam peptide is prepared by adopting the HonDau clam, and is used for relieving the inflammatory reaction of an organism and the damage of renal function caused by hypertension, and is further applied to the inflammation and the damage of kidney caused by other diseases and health care foods with related functions.

### CROSS REFERENCE TO RELATED APPLICATION

This patent application is an international application of PCT/CN2021/116427 with a filing date of Sep. 3, 2021, which claims the benefit and priority of Chinese Patent Application No. CN 202010922422.X filed on Sep. 4, 2020 and the Chinese Patent Application No. CN 202110997320.9 filed on Aug. 27, 2020, the entire contents of which are incorporated by reference herein in its entirety as part of the present application.

### TECHNICAL FIELD

The invention relates to the technical field of biomedicine, and more specifically, to a preparation method and application of an anti-inflammatory kidney protecting clam peptide.

### BACKGROUND ART

In real life, inflammatory response is a moderate or abnormal system response of the body's inflammation-immune related cells according to the changes of internal and external environment. The occurrence of various diseases is related to inflammation, so inflammation is the basic pathological feature of many diseases, which can occur in various tissues and organs, and is also the main link of drug intervention. Inflammation generally leads to changes in the levels of important inflammatory factors such as IL-6, IL-8, TNF-α and CRP. At present, common anti-inflammatory drugs mainly include penicillin, cephalosporin, amoxicillin, etc. Long-term use of such drugs will lead to drug resistance of the body, causing damage to liver, kidney and other functions. The development of food-borne anti-inflammatory substances with less toxic side effects has thus become an urgent need for the majority of patients.

Some diseases can not only cause related inflammatory reactions, but also affect renal function, which are mutually causal and mutually influential, among which hypertension is a disease that can affect both inflammation and renal function. Renal function refers to the function of the kidney to excrete metabolic wastes and maintain the balance of sodium, potassium and calcium in the body. Serum creatinine, urea/urea nitrogen, uric acid and so on are common indicators of renal function. The main manifestations of renal function damage are kidney deficiency, renal insufficiency, renal failure and renal function damage, which can develop into uremia in severe cases and endanger life. At present, that medicine for protecting the renal function on the market are mainly Chinese herbal medicine, which play a role in relieving and protecting, there is no specific drug that can reverse the damage of renal function.

Clam is a common low-value shellfish widely distributed in the coastal areas of China, which has the characteristics of high protein, low fat, high trace elements, low sugar and so on. It is a high-quality marine product with rich nutrition and high therapeutic and medicinal value. The clam peptide belongs to small molecule bioactive peptide of marine source, has high safety, is easy to be absorbed, and has the functions of enhancing immunity. The bioactive peptides from marine clam have become a hot research topic. Most of the studies on bioactive peptides from Mactra veneriformis focus on the biological functions such as lowering blood pressure, reducing blood lipid and delaying aging, but there are few studies on other aspects of bioactive peptides from Mactra veneriformis, and there are no reports about the effects of bioactive peptides on alleviating inflammation caused by hypertension and protecting renal function.

Therefore, in combination with the above problems, it is an urgent problem for those skilled in the art to provide a preparation method of clam peptide that can be applied to anti-inflammatory drugs and drugs for protecting renal function.

### SUMMARY

In view of this, a preparation method and application of an anti-inflammatory and kidney-protecting clam peptide are disclosed. The clam peptide is prepared from HonDau, and is used for alleviating the body inflammatory reaction and kidney function damage caused by hypertension, and is further applied to the inflammation and kidney damage caused by other diseases.

In order to achieve the above purpose, the invention adopts the following technical scheme:

A method for preparing an anti-inflammatory and kidney-protecting clam peptide, which includes the following steps as follows.

Cleaning and mashing whole clam meat to obtain slurry, adding compound protease accounting for 0.1-0.3% of the weight of the slurry, performing enzymolysis, centrifugation, membrane separation and purification, and spray drying to obtain clam peptide powder.

Preferably, the compound protease includes neutral protease, alkaline protease and flavor protease, and the weight ratio of the neutral protease to the alkaline protease to the flavor protease is 2:1:1.

Preferably, the weight ratio of the clam meat to the water during the enzymolysis is 1:1 to 1:3.

Preferably, during the enzymolysis, the clam peptide is enzymolyzed for 4 to 6 hours at a temperature of 50 to 60° C.

Preferably, during the centrifugation, the centrifugal rotation speed of the clam peptide in the production process is 16000 R/min.

Preferably, during the membrane separation and purification, the clam peptide enzymolysis solution is filtered by microfiltration-ultrafiltration-nanofiltration membrane, and the enzymolysis solution with the molecular weight below 2 KDa is intercepted.

An application of an anti-inflammatory and kidney-protecting clam peptide in the preparation of an anti-inflammatory medicament and a medicament for protecting kidney function is also disclosed.

Through the technical scheme, compared with the prior art, the invention has the following beneficial effects:

1. the clam peptide in the disclosure is proved to have obvious effect of inhibiting inflammatory factor through experiments.

2, the clam peptide of the disclosure is proved to have the effect of obviously reducing serum creatinine, uric acid and urea nitrogen in renal function index through experiments.

3. The disclosure expands the application of the clam peptide in medical care, has the advantages of high safety and easy absorption by the human body, and provides a new choice of food-borne medicine for patients to resist inflammation and protect the kidney.

The method for producing the clam peptide provided by the technical scheme has the advantages of simple process, mild condition, short period, no addition of any inorganic or organic solvent, high yield and suitability for industrial production. The prepared clam peptide has pure and white color, no fishy smell, no other peculiar smell, and good taste and flavor. And the molecular weight of the clam active peptide is small, and the clam active peptide is mainly tetrapeptide-hexapeptide, which is easy to be absorbed by human body.

### BRIEF DESCRIPTION OF THE DRAWINGS

In order to more clearly illustrate the embodiments of the present disclosure or the technical solutions in the prior art, the drawings required to be used in the description of the embodiments or the prior art will be briefly described below. Obviously, the drawings in the following description are only the embodiments of the present disclosure and for those of ordinary skill in the art, other figures may also be obtained from the figures provided.

FIG. 1 shows the effect of clam peptide on serum IL-8 in rats;

FIG. 2 shows the effect of clam peptide on TNF-α in rat serum;

FIG. 3 shows the effect of clam peptide on hs-CRP in rat serum;

FIG. 4 shows the effect of clam peptide on SCr in rat serum;

FIG. 5 shows the effect of clam peptide on SUA in rat serum; and

FIG. 6 shows the effect of clam peptide on BUN in rat serum.

### DETAILED DESCRIPTION OF THE EMBODIMENTS

Hereinafter, the embodiments of the disclosure will be described in detail in combination with the examples, so that the experimental process of how to apply the technical means to solve the technical problems and achieve the technical effects of the disclosure can be fully understood and implemented.

The following is a clear and complete description of the technical solutions in the embodiments of the present disclosure. Obviously, the described embodiments are only a part of the embodiments of the disclosure, but not all of them. Based on the embodiments of the disclosure, all other embodiments obtained by a person of ordinary skill in the art without creative effort are within the protection scope of the disclosure.

### Embodiment 1

The embodiment 1 of the disclosure provides a preparation method and application of an anti-inflammatory and kidney-protecting clam peptide, which adopts the technical scheme as follows.

The whole meat of the HonDau clam is prepared, washed, drained and homogenized, and a certain amount of deionized water is added into the clam meat slurry, so that the final weight ratio of the clam meat to the water is 1:2. Enzymatic hydrolysis was carried out at 50° C. for 4 h with 0.13% protease (neutral protease:alkaline protease:flavourzyme=2:1:1), centrifuged at 16000 r/min, and the supernatant was filtered by microfiltration-ultrafiltration-nanofiltration membrane. Spraying and drying to obtain the clam peptide.

### Embodiment 2

The embodiment 2 of the disclosure provides a preparation method and application of an anti-inflammatory and kidney-protecting clam peptide, which adopts the technical scheme as follows.

The whole meat of the HonDau clam is prepared, washed, drained and homogenized, and a certain amount of deionized water is added into the clam meat slurry, so that the final weight ratio of the clam meat to the water is 1:3. Enzymatic hydrolysis was carried out at 50° C. for 6 h with 0.13% protease (neutral protease:alkaline protease:flavourzyme=2:1:1), centrifuged at 16000 r/min, and the supernatant was filtered by microfiltration-ultrafiltration-nanofiltration membrane. Spraying and drying to obtain the clam peptide.

### Embodiment 3

The embodiment 3 of the disclosure provides a preparation method and application of an anti-inflammatory and kidney-protecting clam peptide, and the technical scheme is adopted as follows.

The whole meat of the HonDau clam is provided, washed, drained and homogenized, and a certain amount of deionized water is added into the clam meat slurry, so that the final weight ratio of the clam meat to the water is 1:2. Enzymatic hydrolysis was carried out at 55° C. for 4 h with 0.2% protease (neutral protease:alkaline protease:flavourzyme=2:1:1), centrifuged at 16000 r/min, and the supernatant was filtered by microfiltration-ultrafiltration-nanofiltration membrane. Spraying and drying to obtain the clam peptide.

Anti-inflammatory test after administration in spontaneously hypertensive rats is as follows.

Spontaneously hypertensive rats were purchased from Beijing Weitong Lihua Experimental Animal Technology Co., Ltd.

Forty male spontaneously hypertensive rats (SHR), aged about 12 weeks and weighing about 270 G, were adaptively fed for one week. The rats were randomly divided into 5 groups, including model control group (MC), positive drug control group (AC), low-dose group (CP1), medium-dose group (CP2) and high-dose group of clam peptide (CP3) (low-dose group 50 mg/kg, medium-dose group 100 mg/kg, high-dose group 200 mg/kg) with 8 rats in each group. The rats in the AC group were given captopril 10 mg/kg by gavage, at the same time, the MC group was given the same volume of distilled water. The drug was administered once a day for 4 weeks. Serum IL-8, TNF-α and hs-CRP were measured, and the data were analyzed by SPSS software. The results showed that the mean value of the index was ±standard deviation, p<0.05 indicated significant difference, and p<0.01 indicated very significant difference. The statistical results of the data are shown in Table 1.

TABLE 1
Effect of clam peptide on IL-8, TNF-α and hs-CRP in Hypertensive

| Inflammatory factors | MC | AC | CP1 | CP2 | CP3 |
|---|---|---|---|---|---|
| IL-8(pg/ad) | 181.20 ± 27.72 | 122.00 ± 12.15** | 143.30 ± 17.70** | 158.40 ± 18.52 | 89.08 ± 16.27** |
| TNF-α(pg/ml) | 148.70 ± 52.12 | 87.41 ± 19.19** | 119.80 ± 16.18 | 139.20 ± 8.87 | 84.43 ± 18.95** |
| hs-CRP(μg/L) | 2.16 ± 0.36 | 1.53 ± 0.41* | 1.83 ± 0.36 | 1.75 ± 0.49 | 1.43 ± 0.55** |

Note: Compared with the model group, *: p<0.05; **: p<0.01

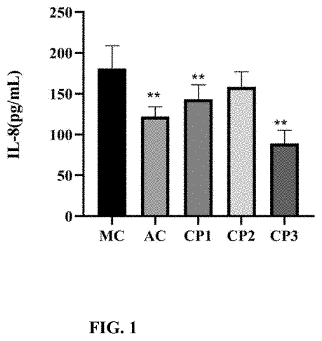

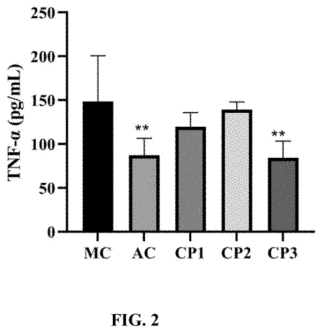

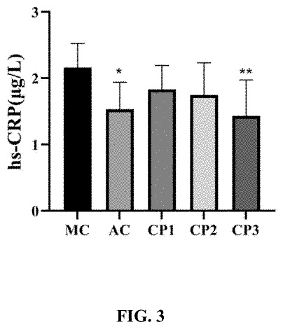

As shown in FIG. 1, the clam peptide group can reduce the level of IL-8, and compared with the model group (the content of IL-8 is 181.2±27.72 PG/ml), the high dose group and the positive drug group reduced the serum IL-8 level to 89.08±16.27 PG/ml (p<0.01) and 122±12.15 PG/ml (p<0.01), respectively, with a statistically significant difference. FIG. 2 shows that the content of TNF-α in the serum of the high-dose clam peptide group is reduced by nearly 43% compared with that of the model group, and has extremely significant difference and achieve the effect equivalent to that of the positive medicine. FIG. 3 reflects the effect of clam peptide on hs-CRP in rat serum. With the increase of clam peptide dose, hs-CRP showed a trend of gradual decrease. Compared with the model control group (2.16±0.36 μg/L), the high-dose clam peptide-infused group decreased to 1.43±0.55 μg/L, which was significantly different and close to the level of the positive drug group.

The above results indicate that the clam peptide can effectively reduce the levels of inflammatory factors such as IL-8, TNF-α and hs-CRP in rat serum, has good anti-inflammatory effect, and can be applied to anti-inflammatory related drugs or health products.

Protection of renal function in spontaneously hypertensive rats after administration of the drug is provided as follows.

Spontaneously hypertensive rats were purchased from Beijing Weitong Lihua Experimental Animal Technology Co., Ltd.

Forty male spontaneously hypertensive rats (SHR), aged about 12 weeks and weighing about 270 G, were adaptively fed for one week. The rats were randomly divided into 5 groups, including model control group (MC), positive drug control group (AC), low-dose group (CP1), medium-dose group (CP2) and high-dose group of clam peptide (CP3) (low-dose group 50 mg/kg, medium-dose group 100 mg/kg, high-dose group 200 mg/kg) with 8 rats in each group. The rats in the AC group were given captopril 10 mg/kg by gavage, At the same time, the MC group was given the same volume of distilled water. The drug was administered once a day for 4 weeks. SCr, SUA and BUN were measured, and the data were analyzed by SPSS software. Analysis of variance was used, and the results were shown as mean±standard deviation of the index, with p<0.05 for significant difference and p<0.01 for very significant difference. The statistical results of the data are shown in Table 2.

TABLE 2
Effect of clam peptide on SCr, SUA and BUN in Hypertensive Rats

| Indicators of renal | MC | AC | CP1 | CP2 | CP3 |
|---|---|---|---|---|---|
| SCr(μmol/L) | 687.10 ± 111.20 | 421.50 ± 101.20** | 508.90 ± 128.90* | 527.60 ± 66.78** | 330.50 ± 75.89** |
| SUA(mg/L) | 283.50 ± 56.66 | 211.60 ± 39.89** | 220.90 ± 37.27* | 245.90 ± 39.17 | 169.90 ± 30.11** |
| BUN(mmol/L) | 17.06 ± 3.70 | 10.56 ± 3.17** | 12.43 ± 2.76** | 13.69 ± 2.29 | 8.10 ± 2.43** |

Note:
Compared with the model group,
*p < 0.05;
**p < 0.01

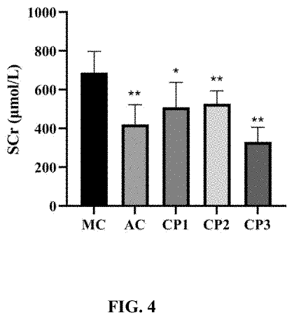

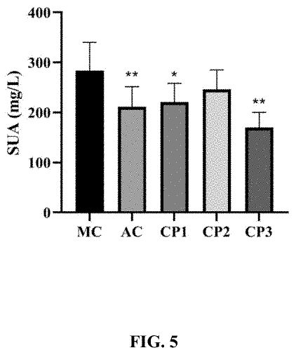

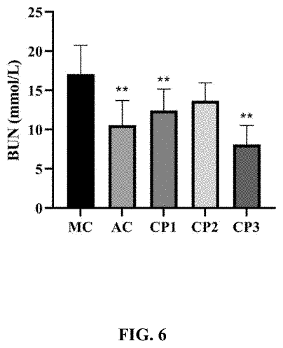

According to FIG. 4, different doses of clam peptide can reduce the serum SCr of rats, and the high dose group has the most significant effect, which can be reduced by 51.9% compared with the model control group. FIG. 5 shows that clam peptide has the effect of reducing SUA. Compared with the model control group (283.50±56.66 mg/L), the high dose group reduces SUA to 169.90±30.11 mg/L, which has a very significant difference. As can be seen from FIG. 6, the clam peptide has a good effect on the clearance of BUN in serum, and the effect of high dose group of clam peptide is the most significant, compared with the model control, the clearance rate of BUN can reach 52.5%.

The above results show that the indexes of SCr, SUA and BUN which affect the renal function in the gavage group of the clam peptide are significantly reduced, and the clam peptide has a certain protective effect on the kidney of the rat, so that the clam peptide has the activity of protecting the renal function and can be used in medicines or health care product related to renal function protection.

Food test for patients with hypertension is as follows.

Ten patients with hypertension were selected, and on the basis of taking the original antihypertensive drug unchanged, 10 patients were additionally given clam peptide Capsules (prepared from the finished product of embodiment 1, 300 mg/capsule) twice a day, once in the morning and once in the evening, 3 capsules each time, half an hour after breakfast/dinner, before 9: During the trial period, you can choose to stop taking the original antihypertensive drugs according to your physical condition and blood pressure changes. Intervention for 90 days (3 months). During the study, the patients' blood pressure was measured every day, Duchenne hypertension quality of life and 36-item simple health survey were scored every two weeks/month, and physiological, biochemical and safety evaluation indicators were detected before treatment, 30 days (one month) and 90 days (three months) to observe and compare the changes of patients' indicators and evaluate the effectiveness and safety of clam peptide. The data were analyzed by SPSS.

The statistics of relevant data results are as follows:

TABLE 3
SBP (Systolic Blood Pressure) (^{x} ± s, mmHg, n = 10)

| | 0 d | 1-7 d | 8-14 d | 15-21 d | 22-30 d | 31-37 d | 38-44 d | 45-51 d |
|---|---|---|---|---|---|---|---|---|
| SBP | 144.16 ± | 143.80 ± | 140.66 ± | 136.63 ± | 135.88 ± | 135.36 ± | 134.24 ± | 134.49 ± |
| (systolic | 9.83 | 11.62 | 9.48 | 5.97* | 7.98** | 6.81** | 9.17** | 6.22** |
| blood | | | | | | | | |
| pressure) | | | | | | | | |

| | | | | | | Follow-up | Follow-up |
|---|---|---|---|---|---|---|---|
| | 52-60 d | 61-67 d | 68-74 d | 75-81 d | 82-90 d | 1-7 d | 8-14 d |
| SBP | 133.00 ± | 131.33 ± | 132.34 ± | 130.59 ± | 130.40 ± | 130.63 ± | 129.37 ± |
| (systolic | 6.79*** | 8.16*** | 7.26*** | 9.06*** | 8.12*** | 8.25** | 9.30*** |
| blood | | | | | | | |
| pressure) | | | | | | | |

Note:
*p < 0.05,
**p < 0.01,
***p < 0.001 compared to 0 d

It can be seen from Table 3 that \textcircled{1} SBP decreased continuously during the test; \textcircled{2} Compared with 0 day, there was a significant difference (p<0.05) after taking 21 days (3 weeks), a significant difference (p<0.01) after taking 30 days (1 month), and a very significant difference after taking 60 days (2 months). \textcircled{3} After 81 days (about 3 months) of administration, SBP decreased to normal level, and remained at normal level after 14 days (2 weeks) of follow-up; \textcircled{4} After administration for 14 days (2 weeks), blood pressure was reduced from grade 1 hypertension to high normal blood pressure, and after 81 days (about 3 months), blood pressure was reduced from high normal blood pressure to normal blood pressure.

TABLE 4
DBP (Diastolic Blood Pressure) (^{x} ± s, mmHg, n = 10)

| | 0 d | 1-7 d | 8-14 d | 15-21 d | 22-30 d | 31-37 d | 38-44 d | 45-51 d |
|---|---|---|---|---|---|---|---|---|
| DBP | 88.13 ± | 87.19 ± | 85.11 ± | 82.50 ± | 82.27 ± | 81.96 ± | 81.04 ± | 80.09 ± |
| (Diastolic | 6.58 | 7.14 | 6.32 | 5.82* | 8.42* | 6.20** | 7.02** | 6.06** |
| Blood | | | | | | | | |
| Pressure) | | | | | | | | |

| | | | | | | Follow- | Follow- |
|---|---|---|---|---|---|---|---|
| | 52-60 d | 61-67 d | 68-74 d | 75-81 d | 82-90 d | up 1-7 d | up 8-14 d |
| DBP | 80.41 ± | 79.39 ± | 79.23 ± | 80.49 ± | 79.99 ± | 78.90 ± | 78.21 ± |
| (Diastolic | 5.40** | 6.43*** | 4.60*** | 5.54*** | 5.34*** | 5.52*** | 6.57*** |
| Blood | | | | | | | |
| Pressure) | | | | | | | |

Note:
*p < 0.05,
**p < 0.01,
***p < 0.001 compared to 0 d

It can be seen from Table 4 that \textcircled{1} DBP decreased continuously during the test; \textcircled{2} Compared with 0 day, there was a significant difference (p<0.05) after taking 21 days (3 weeks), a significant difference (p<0.01) after taking 37 days (5 weeks), and a very significant difference after taking 60 days (2 months). \textcircled{3} DBP decreased to normal level after 14 days (2 weeks) of treatment, and DBP remained at normal level after 14 days (2 weeks) of follow-up; \textcircled{4} After administration for 14 days (2 weeks), blood pressure decreased from high normal to normal.

The blood pressure was graded and compared, and it was found that after 14 days of administration, grade 2 hypertension was converted to grade 1 hypertension, and part of normal high blood pressure was converted to normal blood pressure. There was no significant difference in blood pressure between day 0 and days 1-7 and 15-21 (p>0.05). Compared with the blood pressure on the 8th to 14th and 22nd to 44th day, there was a significant difference (p<0.05). There was a significant difference (p<0.01) compared with the blood pressure at 45 to 90 days after the trial and at 14 days after the follow-up. During the experimental period, the blood pressure of two of them fluctuated all the time. In order to maintain the authenticity and integrity of the data, the data were not excluded during the analysis. Therefore, the blood pressure rose after 68-90 days of trial, which was speculated to be related to the diet and physical condition of the trial personnel.

TABLE 5
Occupancy rate of people with blood pressure classification

| | | Normal | Normal | | |
|---|---|---|---|---|---|
| Trial | | blood | High | Grade 1 | Grade 2 |
| feeding time | | pressure | Value | hypertension | hypertension |
| 0 | d | 0.00% | 30.00% | 60.00% | 10.00% |
| 1-7 | d | 0.00% | 40.00% | 50.00% | 10.00% |
| 8-14 | d | 20.00% | 20.00% | 60.00% | 0.00% |
| 15-21 | d | 10.00% | 50.00% | 40.00% | 0.00% |
| 22-30 | d | 30.00% | 30.00% | 40.00% | 0.00% |
| 31-37 | d | 30.00% | 40.00% | 30.00% | 0.00% |
| 38-44 | d | 30.00% | 40.00% | 30.00% | 0.00% |
| 45-51 | d | 30.00% | 60.00% | 10.00% | 0.00% |
| 52-60 | d | 20.00% | 70.00% | 10.00% | 0.00% |
| 61-67 | d | 30.00% | 70.00% | 0.00% | 0.00% |
| 68-74 | d | 40.00% | 40.00% | 20.00% | 0.00% |
| 75-81 | d | 30.00% | 60.00% | 10.00% | 0.00% |
| 82-90 | d | 40.00% | 50.00% | 10.00% | 0.00% |

| | | | | |
|---|---|---|---|---|
| Follow-up | 40.00% | 60.00% | 0.00% | 0.00% |
| 1-7 d | | | | |
| Follow-up | 40.00% | 60.00% | 0.00% | 0.00% |
| 8-14 d | | | | |

The above result show that that clam peptide can significantly reduce the blood pressure (systolic blood pressure and diastolic blood pressure) of patients with hypertension, has good antihypertensive effect, and can be apply to antihypertensive related medicines or health care products.

TABLE 6
Comparison of Duchenne hypertension quality of life scores (^{x} ± s, min)

| Project | | | | | |
|---|---|---|---|---|---|
| Project Time | | | | | |
| Project Think, | | | | | |
| Xian | 0 d | 14 d | 30 d | 60 d | 90 d |
| Physiologic condition | 73.60 ± 6.06 | 75.90 ± 4.61 | 76.70 ± 4.79 | 76.50 ± 5.13* | 78.50 ± 4.55** |
| Somatization symptom | 25.50 ± 2.01 | 25.90 ± 1.60 | 26.50 ± 0.97 | 26.50 ± 2.07 | 27.20 ± 0.92* |
| Sexual dysfunction | 5.30 ± 1.42 | 5.60 ± 1.65 | 5.90 ± 1.52 | 6.40 ± 1.26* | 6.70 ± 1.25* |
| Sleep status | 10.70 ± 1.25 | 10.50 ± 1.35 | 10.90 ± 1.20 | 11.10 ± 0.88 | 11.20 ± 1.03 |
| Vitality or vitality. | 6.50 ± 0.85 | 6.90 ± 0.57 | 6.90 ± 0.88 | 7.10 ± 0.88 | 7.40 ± 0.70* |
| Anxiety | 10.90 ± 1.20 | 11.60 ± 0.70 | 11.70 ± 0.67 | 11.60 ± 0.52 | 11.90 ± 0.32* |
| Repression | 15.50 ± 0.97 | 15.80 ± 0.42 | 15.80 ± 0.42 | 15.80 ± 0.42 | 15.90 ± 0.32 |
| Forced condition | 10.70 ± 1.70 | 10.70 ± 1.64 | 10.90 ± 1.45 | 11.30 ± 1.16 | 11.50 ± 1.08 |
| Interpersonal | 15.10 ± 0.74 | 15.30 ± 0.48 | 15.30 ± 0.82 | 15.40 ± 0.97 | 15.70 ± 0.48* |
| sensitivity | | | | | |
| Working Status | 7.00 ± 1.05 | 7.60 ± 0.52 | 7.50 ± 0.71 | 7.50 ± 0.71 | 7.50 ± 0.71 |
| Hostile | 11.10 ± 0.74 | 11.50 ± 0.53 | 11.70 ± 0.48* | 11.60 ± 0.70 | 11.80 ± 0.63** |
| Total Score | 203.00 ± 14.64 | 208.70 ± 10.81 | 211.30 ± 11.48 | 212.40 ± 11.74 | 217.00 ± 9.98* |

Note:
*p < 0.05,
**p < 0.01 compared to 0 d

According to Table 6, compared with 0 day, the total score of Duchenne hypertension quality of life scale increased after taking clam peptide, and there was a statistical difference (p<0.05) after taking clam peptide for 90 days (3 months). The scores of physiological status, somatization symptoms, sexual dysfunction, sleep status, anger or vitality, anxiety, depression, obsessive-compulsive status, interpersonal sensitivity, work status and hostility were increased. After 30 days (1 month), there was a significant difference in the dimension of hostility (p<0.05). After 60 days (2 months), there were significant differences in the dimensions of physiological status and sexual dysfunction (p<0.05). After 90 days (3 months), there were significant differences in somatization symptoms, sexual dysfunction, anger or vitality, anxiety and interpersonal sensitivity (p<0.05), and there were significant differences in physiological status and hostility (p<0.01).

TABLE 7
Comparison of 36-item simple health survey scores (^{x} ± s, min)

| Project Time | 0 d | 14 d | 30 d | 60 d | 90 d |
|---|---|---|---|---|---|
| Physiological function PF | 84.00 ± 10.75 | 84.50 ± 17.71 | 89.00 ± 9.66 | 89.50 ± 9.56 | 89.00 ± 12.65 |
| Physiological function RP | 85.00 ± 24.15 | 100.00 ± 0.00 | 100.00 ± 0.00 | 95.00 ± 15.81 | 100.00 ± 0.00 |
| Somatic pain BP | 83.80 ± 9.59 | 84.00 ± 11.04 | 87.80 ± 14.22 | 87.40 ± 12.51 | 96.80 ± 6.75* |
| General Health Status GH | 77.50 ± 13.79 | 77.20 ± 8.66 | 83.60 ± 10.71 | 80.90 ± 13.96 | 88.30 ± 10.23* |
| Energy VT | 86.50 ± 11.32 | 93.00 ± 9.78* | 90.50 ± 13.43 | 93.00 ± 7.89 | 89.00 ± 17.61 |
| Social function SF | 120.00 ± 8.74 | 125.00 ± 0.00 | 123.75 ± 3.95 | 116.25 ± 20.45 | 106.25 ± 30.19 |
| Affective function RE | 86.67 ± 28.11 | 96.67 ± 10.54 | 96.67 ± 10.54 | 96.67 ± 10.54 | 100.00 ± 0.00 |
| Mental Health MH | 84.80 ± 10.80 | 85.20 ± 9.44 | 86.80 ± 9.44 | 88.40 ± 12.71 | 86.00 ± 16.68 |
| Health Change HT | 47.50 ± 21.89 | 50.00 ± 20.41 | 52.50 ± 24.86 | 57.50 ± 26.48 | 60.00 ± 29.34 |
| Total Score | 755.77 ± 76.95 | 795.57 ± 32.00 | 810.62 ± 53.50* | 804.62 ± 59.69 | 815.35 ± 63.30 |

Note:
*p < 0.05 compared to 0 d

It can be seen from Table 7 that, compared with 0 day, the total score of 36-item simple health questionnaire increased after taking clam peptide, and there was a statistical difference (p<0.05) after taking clam peptide for 30 days (1 month). The scores of physiological function, physiological function, body pain, general health status, energy, emotional function, mental health and health change increased. After 14 days of administration (2 weeks), there was a significant difference in the energy dimension (p<0.05). After 90 days (3 months), there were significant differences in the dimensions of body pain and general health (p<0.05). The dimension of social function showed a trend of increasing first and then decreasing, but there was no significant difference (p>0.05).

The above results show that the quality of life and physical condition of patients with hypertension can be significantly improved by taking clam peptide.

TABLE 8
Relevant influencing factors of heart function,
liver function and kidney function

| Factor Time/d | 0 | 30 | 90 |
|---|---|---|---|
| Aspartate | 19.54 ± 5.13 | 15.71 ± 4.03** | 19.26 ± 4.66 |
| aminotransferase | | | |
| (AST) (U/L) | | | |
| CK-Mb (M) | 3.25 ± 0.72 | 3.45 ± 0.64 | 1.74 ± 0.60*** |
| (ng/mL) | | | |
| CREA | 81.20 ± 9.00 | 73.70 ± 9.18*** | 73.50 ± 12.14*** |
| (μmol/L) | | | |

Note:
**p < 0.01,
***p < 0.001 compared to 0 d

It can be seen from Table 8 that, compared with day 0, the content of glutamic oxaloacetic transaminase, creatine phosphokinase isoenzyme and creatinine in patients with hypertension can be reduced by taking clam peptide. After 30 days (1 month), there was a significant difference in glutamic oxaloacetic transaminase (p<0.01) and creatinine (p<0.001). There was a significant difference (p<0.001) in creatine phosphokinase isoenzyme and creatinine after 90 days (3 months) of administration.

TABLE 9
Blood pressure lowering factors

| Factor Time/d | 0 | 30 | 90 |
|---|---|---|---|
| Endothelin-1 | 36.55 ± 45.10 | 3.58 ± 0.19* | 19.02 ± 14.53 |
| (ET-1) PG/mL | | | |
| Angiotensin I | 469.54 ± 111.77 | 461.50 ± 151.55 | 446.20 ± 121.64 |
| converting enzyme | | | |
| (ace I) (ng/mL) | | | |
| Angiotensin | 0.61 ± 0.06 | 0.60 ± 0.04 | 0.79 ± 0.15* |
| converting enzyme 2 | | | |
| (ACE2) (ng/mL) | | | |

Note:
*p < 0.05 compared to 0 d

It can be seen from Table 9 that compared with day 0, the administration of clam peptide can reduce the content of endothelin and angiotensin I converting enzyme in patients with hypertension, and increase the content of angiotensin converting enzyme 2. After 30 days (1 month), there was a significant difference in endothelin (p<0.05). There was a statistical difference (p<0.05) for ACE2 at 90 days (3 months).

TABLE 10
Vascular endothelial factor

| Factor Time/d | 0 | 30 | 90 |
|---|---|---|---|
| Nitric oxide | 56.90 ± 21.86 | 46.80 ± 17.55 | 63.64 ± 14.82 |
| (μmol/L) | | | |

From Table 10, it can be seen that compared with 0 day, the content of nitric oxide in hypertensive patients can be increased by taking clam peptide for 90 days (3 months), but there is no statistical difference (p>0.05).

TABLE 11
Inflammatory factors

| Factor Time/d | 0 | 30 | 90 |
|---|---|---|---|
| Interleukin-1β | 2243.38 ± 3286.85 | 131.08 ± 98.51 | 1266.99 ± 1045.32 |
| (IL-1β) (PG/mL) | | | |
| Interleukin 17A | 106.54 ± 132.37 | 16.53 ± 0.89 | 42.04 ± 17.96 |
| (IL-17a) (PG/mL) | | | |
| Tumor necrosis | 49.56 ± 59.4 | 15.32 ± 3.41 | 24.22 ± 21.71 |
| factor alpha | | | |
| (TNF-α) (PG/mL) | | | |
| IL-10 (PG/mL) | 356.22 ± 610.36 | 23.03 ± 1.45 | 444.28 ± 166.05 |

It can be seen from Table 11 that compared with 0 day, the content of interleukin 1 beta, interleukin 17A and tumor necrosis factor alpha in patients with hypertension can be reduced by taking clam peptide, while the content of interleukin 10 can be increased, but there is no statistical difference (p>0.05).

TABLE 12
Oxidative stress factors

| Factor Time/d | 0 | 30 | 90 |
|---|---|---|---|
| Malonaldehyde | 42.29 ± 9.35 | 37.40 ± 10.50 | 30.91 ± 4.49** |
| (MDA) (μmol/L) | | | |

Note:
**p < 0.01 compared to 0 d

It can be seen from Table 12 that, compared with 0 day, the content of malondialdehyde in the hypertensive patients was reduced after taking the clam peptide, and there was a significant difference (p<0.01) after taking the clam peptide for 90 days (3 months).

The results showed that the contents of AST, CK-Mb (M), CREA, ET-1, ACEI, IL-1β, IL-17a, TNF-α and MDA were decreased, and the contents of ACE2, NO and IL-10 were increased in patients with hypertension after taking clam peptide. On the one hand, it suggests that taking clam peptide is helpful to improve the heart, liver and kidney functions of patients with hypertension. On the other hand, the hypotensive mechanism of the clam peptide may be related to the regulation of the content of hypotensive factors and vascular endothelial factors, the reduction of inflammatory reaction and the improvement of antioxidant capacity, and the clam peptide can be applied to medicines or health products related to the reduction of inflammatory reaction and renal function damage caused by hypertension or other diseases.

The embodiments in this specification are described in a progressive manner, and each embodiment focuses on the differences from other embodiments, and the same and similar parts among the embodiments are referred to each other.

The foregoing description of the disclosed embodiments will enable any person skilled in the art to make or use the disclosure. Various modifications to these embodiments will be readily apparent to those skilled in the art, and the generic principles defined herein may be applied without departing from the spirit or scope of the invention.

What is claimed is:

1. A method for preparing an anti-inflammatory and kidney-protecting clam peptide, comprising:
cleaning and mashing a whole clam meat to obtain slurry;
adding compound protease accounting of 0.1-0.3% of a weight of the slurry; and
performing enzymolysis, centrifugation, membrane separation and purification, and spray drying to obtain clam peptide powder.

2. The method of claim 1, wherein the compound protease comprises neutral protease, alkaline protease and flavor protease; and the weight ratio of the neutral protease to the alkaline protease to the flavor protease is 2:1:1.

3. The method of claim 1, wherein the weight ratio of the clam meat to water is 1:1 to 1:3 during the enzymolysis.

4. The method of claim 1, wherein during the enzymolysis, the clam peptide is subjected to enzymolysis at a temperature of 50-60° C. for 4-6 hours.

5. The method of claim 1, wherein during the centrifugation, a centrifugal speed of the clam peptide in the production process is 16000 R/min.

6. The method of claim 1, wherein during the membrane separation and purification, the clam peptide enzymolysis solution is subjected to microfiltration-ultrafiltration-nanofiltration membrane filtration, and the enzymolysis solution with a molecular weight below 2 KDa is intercepted.

7. An application of an anti-inflammatory and kidney-protecting clam peptide in the preparation of an anti-inflammatory drug and a drug for protecting renal function.
